(19) United States
(12) Patent Application Publication
(10) Pub. No.: US 2019/0110749 A1
(43) Pub. Date: Apr. 18, 2019

(54) SECURING PATCH FOR WEARABLE MEDICAL DEVICE

(71) Applicant: Jason Forrester, St. Augustine, FL (US)

(72) Inventor: Jason Forrester, St. Augustine, FL (US)

(21) Appl. No.: 16/158,954

(22) Filed: Oct. 12, 2018

Related U.S. Application Data
(60) Provisional application No. 62/571,476, filed on Oct. 12, 2017.

(51) Int. Cl. A61B 5/00; A61B 5/145; A61K 9/70

(52) U.S. Cl. CPC A61B 5/6833; A61B 5/1451; A61K 9/703; A61B 2562/164; A61B 2560/0443; A61B 5/0002; A61B 5/14532

(57) ABSTRACT

The present disclosure provides for methods and an apparatus to provide an adhesive securing patch to keep a monitoring system or other apparatus pressed against a body part. Generally, a wearable medical device is pressed against or into a patient. A first flexible substrate layer is applied to the wearable medical device to secure it against the patient's skin. The flexibility of this substrate layer creates a cavity for any supracutaneous portion of the medical device. A second adhesive substrate layer is applied to secure the first substrate layer, as well as to provide a visual display, such as medical information or an image. A third, larger, transparent substrate layer is applied to secure the first and second substrate layers and to allow for exterior viewing of the visual display of the second substrate layer.

### FIELD OF THE DISCLOSURE

The present disclosure relates to methods and apparatus to provide an adhesive securing patch to keep a monitoring system or other wearable medical device pressed against a body part.

### BACKGROUND OF THE DISCLOSURE

Diabetes mellitus is a disorder in which the pancreas cannot create sufficient insulin (Type I or insulin dependent) and/or in which insulin is not effective (Type 2 or noninsulin dependent). In the diabetic state, the victim suffers from high blood sugar, which can cause an array of physiological derangements associated with the deterioration of small blood vessels, for example, kidney failure, skin ulcers, or bleeding into the vitreous of the eye. A hypoglycemic reaction (low blood sugar) can be induced by an inadvertent overdose of insulin, or after a normal dose of insulin or glucose-lowering agent accompanied by extraordinary exercise or insufficient food intake.

Continuous glucose monitoring (CGM) systems are known in the art. For example, a system for inserting a transcutaneous analyte sensor into a host (e.g., a person's body part) may include a transcutaneous analyte sensor for measuring an analyte concentration in the host, and a housing configured for placement adjacent to a skin of the host and configured for receiving at least a portion of the sensor. The system may include further a needle configured to insert the sensor through the housing and into the host, and an applicator configured to mate releasably with the housing. The applicator may include a plunger configured to push the needle and the sensor through the housing into the host. An example of such a CGM system is the DexCom® models G4 and G5, such as described in U.S. Pat. No. 7,310,544.

Sensors of CGM systems of the known art are held in place with adhesive tape or the like. The sensor measures changes in glucose levels in the body's fluid (i.e., interstitial fluid) around the sensor. The sensor is coupled directly to a transmitter, and the transmitter transmits information from the sensor to a monitor, typically by use of a wireless link such as Bluetooth™. The monitor may be implemented as a smart phone app. Each sensor usually is changed every few days, according to each device manufacturer's recommendations.

CGM sensors of the known art need to be held continuously against a user's skin. A medical grade adhesive or adhesive tape is usually used, such as the Dow Corning® MG 7-1010 Soft Skin Adhesive, or the 3M Transpore™ Surgical Tape. However, the adhesive or adhesive tape of the known art is inadequate. For example the adhesive or adhesive tape may be less effective or lose its adhesiveness when exposed to water, which makes it difficult to keep the site clean. The adhesive or adhesive tape may have a short useful lifetime, and may cost about $60 per sensor and may involve a trip to a doctor's office to inspect and replace. Patient movement or movement of the sensor (e.g., if a patient bumps the sensor against something) may degrade the effectiveness of the sensor.

The adhesive or adhesive tape is prone to become loose over time, and the adhesive or adhesive tape does not offer any substantial protection against physical damage. Furthermore, a first responder or a caregiver of a patient may be unfamiliar with the CGM sensor, and if the patient is incapacitated or otherwise unable to cooperate meaningfully and thus cannot assist a caregiver (e.g., a patient who is a child, an elderly patient, a sedated or unconscious patient, a mentally challenged patient, etc.). Consequently, the first responder or caregiver may not take proper care of the CGM sensor, or may not aware of how to respond to problematic readings from the CGM sensor.

Therefore, what is needed is an improved adhesive or adhesive tape that addresses the shortcomings identified above.

### SUMMARY OF THE DISCLOSURE

Embodiments in accordance with the present disclosure methods and apparatus to provide a securing patch for a skin-mounted wearable medical device, such as a subcutaneous or transdermal drug or therapy delivery device, along with electronics associated with and positioned next to the delivery device. The securing patch may be decorated with a pleasing visual or tactile pattern.

Some embodiments include method steps for securing a wearable medical device against or into a patient's skin. The method may include: a) pressing a wearable medical device including a generally planar surface against a surface of skin of a patient; b) fixing the generally planar surface against the surface of skin of the patient with a first substrate layer, where the first substrate layer includes a flexible material and has a surface area sufficiently large to encompass the wearable medical device; and c) securing the first substrate layer to the surface of skin of the patient using a second substrate layer. The second substrate layer may include an adhesive coating and at least one medical information quantum and has a surface area between approximately 3.0 square inches and 4.0 square inches. A medical quantum is operative to convey medical information.

Implementations may include a Radio Frequency Identification (“RFID”) device as a medical information quantum. The method may further include the steps of: scanning the RFID chip using a smart device. The smart device may include a processor; software executable on command; and a display in logical connection with the processor. The smart device may receive RFID chip information; and display the RFID chip information on the display.

The RFID chip information may include patient identification, where the patient identification includes one or more of: a name of the patient, an address of the patient, or a medication list of the patient. The RFID chip information may also include device identification, where the device identification includes one or more of: a serial number of the wearable medical device, a manufacturer of the wearable medical device, a time of installation of the wearable medical device, a geographic location of installation of the wearable medical device, identification information related to an installer of the wearable medical device, an expiration date of the wearable medical device, or a recommended time of removal of the wearable medical device.

In some embodiments, the RFID chip information includes measurements from the wearable medical device, where the measurements include one or more of: an amount of a chemical in the patient's body, an amount of medicine administered to the patient over a defined interval of time through the wearable medical device, or a pulse. The RFID chip information may additionally include a uniform resource locator pointing to a website.

The second or third substrate layer may include a decorative printing on a face of the second substrate layer and a securing a third substrate layer may be secured to the second substrate layer and include an adhesive and has a surface area of between approximately 4.0 square inches and 11.0 square inches.

The third substrate layer preferably includes a water-impermeable barrier. The second or third substrate layer may also include a written message on a surface of the second substrate layer. The written message may include one or more of: a public service message, an advertisement, an instruction about usage of the wearable medical device, a date of installation or first use of the wearable medical device, a service date or expiration date of the wearable medical device, an identification of the patient, a caregiver, an emergency point of contact, a next of kin, or a listing of medical alert information about the patient. The second substrate or third layer may additionally include a decorative printing on a face of the second substrate layer.

In preferred embodiments, the wearable medical device is an insulin pump that includes a subcutaneous probe. The method further includes inserting the subcutaneous probe into the patient's skin prior to the step of securing the first substrate layer.

In some implementations include a method a wearable medical device with a securing patch. The method including the steps of: a) selecting a site on a patient's body, where the site includes a surface area of at least 4.0 square inches; b) affixing the wearable medical device to the site on the patient's body; c) placing a securing patch on the wearable medical device, where the securing patch includes a protective film covering an adhesive, a writable portion, and a hollow chamber of sufficient depth to enclose any supracutaneous portion of the wearable medical device; d) pressing the securing patch to the site; e) holding the securing patch on the site while removing the protective film; f) curing the adhesive; g) applying heat and/or pressure to any uncured portion of the adhesive; and h) placing one or more medical information quanta on the writable portion.

Implementations may include one or more of: the adhesive includes a heat-activated adhesive with a curing temperature between 90.0° Fahrenheit and 101.0° Fahrenheit; the securing patch further includes a design portion, where the design portion includes one or more of: a graphic image, a comic character, a caricature, a sports team logo, or a symbol; and the medical information quanta includes one of: a textual message, an instruction, an advertisement, an identification of the patient, a caregiver, an emergency point of contact, a next of kin, a listing of medical alert information about the patient, or a legal notice.

### BRIEF DESCRIPTION OF THE DRAWINGS

The accompanying drawings, that are incorporated in and constitute a part of this specification, illustrate several embodiments of the disclosure and, together with the description, serve to explain the principles of the disclosure:

FIG. 1 illustrates a comparison of the background art to an embodiment in accordance with the present disclosure; and

FIG. 2 illustrates a method, in accordance with an embodiment of the present invention.

The drawings are not necessarily drawn to scale unless clearly indicated otherwise.

### DETAILED DESCRIPTION

In the following sections, detailed descriptions of examples and methods of the disclosure will be given. The description of both preferred and alternative examples though through are exemplary only, and it is understood that to those skilled in the art that variations, modifications, and alterations may be apparent. It is therefore to be understood that the examples do not limit the broadness of the aspects of the underlying disclosure as defined by the claims.

Medical-grade adhesive tapes or securing patches (collectively, “securing patches”) are known in the background art for securing small sensors, drug delivery devices, and associated small electronics that may be directly coupled to the small sensors or drug delivery devices. The background art uses a general purpose medical-grade tape, or attempts to minimize size of a securing patch so that the securing patch is less noticeable, can be hidden more easily under clothing, produces less noticeable bulges under clothing, affects less skin surface area (such as to allow for more bending near a joint, or to allow for perspiration closer to the patch, etc.) and so forth. With a smaller patch, a stronger adhesive must be used because of fewer square inches of skin contact. A stronger adhesive may be harder to remove, and may have a higher likelihood of painfully pulling out body hairs and/or bruising or rupturing skin, when the adhesive patch is removed.

In contrast, embodiments in accordance with the present disclosure may provide a larger patch over a small sensor, a drug delivery device, and associated small electronics (collectively, “wearable medical devices”). Compared to the background art of medical-grade adhesive tapes or securing patches, embodiments are larger than would be necessary merely to secure a wearable medical device of a predetermined size and weight to a patient's skin. The larger size allows the patch to include highly visible decorative designs or information content, either as printed or embossed on the patch, or by a shape or texture of the patch itself, or a combination thereof. Compared to the background art, where a background art securing patch is something to be hidden or to be unobtrusive, a securing patch in accordance with an embodiment of the present invention allows for a patient to embrace the need to wear a securing patch, by adding a decorative utility, cosmetic utility, or informative utility to the securing patch in addition to a securing utility. The decorative utility draws attention to the securing patch, in contrast to the background art. The cosmetic utility provides a temporary artistic design that the patient may find to be desirable to be on their person, similar to a henna tattoo. Informative content provides information if the patient is unable to provide the information personally, such as, for example, to a caregiver for a minor child or an elderly person with dementia.

Securing patch embodiments in accordance with the present disclosure may include three components or features. First, a flexible transparent protective film may be included. The transparent protective film forms an inner layer of the securing patch, and protects a wearable medical device underneath from damage from exterior forces and/or from other layers of the securing patch.

Second, an open chamber is formed or included in the transparent protective film. The chamber is open on a side of the transparent protective film that is to be facing the wearable medical device. The chamber is a void that is sized sufficiently large to enclose partially the wearable medical device (e.g., enclosed along the top and lateral sides of the wearable medical device, while leaving exposed a bottom side of the wearable medical device so the bottom side can press against a patient's body), but small enough that the wearable medical device can be held snugly against the patient's body without movement of the wearable medical device within the void. In some embodiments, the chamber is sufficiently large to enclose more than half of the wearable medical device.

Embodiments may be provided in a variety of sizes and/or shapes in order to accommodate wearable medical devices of a variety of sizes and shapes. The wall of the chamber should be resistant to punctures and tears. The wall of the chamber may have a stiffness that is balanced between being stiff enough to protect the wearable medical device underneath, and being pliable enough so that the securing patch is not uncomfortable to the patient as the patient moves while wearing the securing patch. In some embodiments, the stiffness of a chamber for a particular securing patch may be set when the securing patch is manufactured (e.g., by selecting a wall thickness or a material composition), or may be selected from a predetermined range of stiffnesses or from a set of stiffness values.

Third, the securing patch may include an outer securing layer, which adhesively secures the entire securing patch to the patient. Typically the outer securing layer will be significantly larger than securing patches of the background art, e.g., at least about twice the surface area of the background art. The large size facilitates the outer securing layer to include a decorative design, custom printing or the like, which is highly visible when the securing patch is worn by the patient. In contrast to the background art, for which a securing patch is designed to be unobtrusive, the securing patch of the present embodiments is designed to be big and bold, and allows a patient to combine the functionality of a securing patch with a decorative aspect.

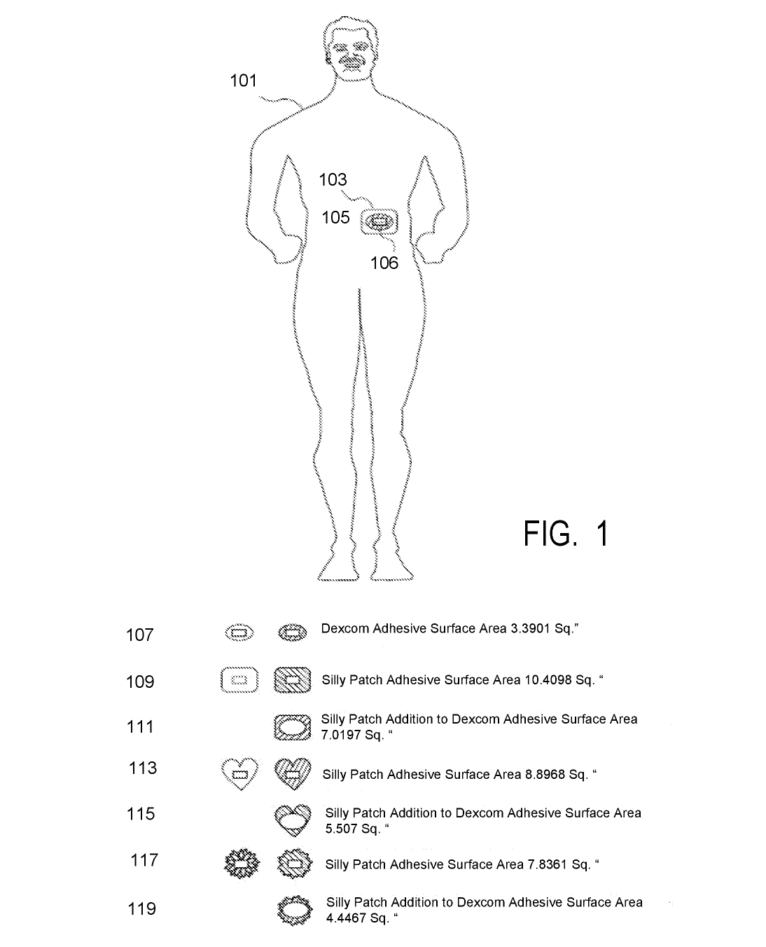

FIG. 1 illustrates a comparison of the background art to an embodiment in accordance with the present disclosure. FIG. 1 illustrates a human 101 who is wearing both a prior art securing patch 105, and a new patch 103 which is an embodiment of the invention. In normal usage, new patch 103 encloses wearable medical device 106 and overlays prior art securing patch 105, without a need to remove prior art securing patch 105. As can be seen, new patch 103 is significantly larger than prior art securing patch 105.

Lines 107, 109 and 111 illustrate a numeric comparison of sizes of both prior art securing patch 105 and new patch 103. The specific sizes listed are merely exemplary in nature. As shown by line 107, prior art securing patch 105 may have an adhesive surface area size of about 3.4 square inches (i.e., between approximately 3.0 square inches and 4.0 square inches), not including the size of wearable medical device 106. In contrast, new patch 103 may have an adhesive surface area size of about 10.4 square inches (i.e., between approximately 4.0 square inches and 11.0 square inches), not including the size of wearable medical device 106. The difference, as illustrated in line 111, means that about 7.0 square inches of additional adhesive surface area is available to affix new patch 103 to human 101, compared to prior art securing patch 105. As noted earlier, a larger size facilitates the outer securing layer to include a decorative design, custom printing or the like, which is designed to be highly visible when the securing patch is worn by the patient.

The size and shape of new patch 103 is exemplary, and other sizes and shapes may be used. For example, FIG. 1 lines 113, 115 illustrate a similar size comparison when new patch 103 has a heart shape, and lines 117, 119 illustrate a similar size comparison when new patch 103 has a star shape.

In some embodiments, new patch 103 may include a hypoallergenic pressure sensitive adhesive medical device tape made with an acrylate adhesive that is safe on skin while being strong and water resistant. The adhesive may be set and cured by application of heat. For example, body heat may be relied upon to set and cure the adhesive (in embodiments in which the curing temperature of the adhesive is approximately human body temperature, or between 90.0° Fahrenheit and 101.0° Fahrenheit), or an external source of heat (e.g., heat lamp, hair dryer, etc.) may be used if faster setting and curing is desired, or if the patient has a condition such as poor circulation that may make it not feasible to rely upon body heat.

Embodiments in accordance with the present disclosure are usable with substantially any type of transdermal or subcutaneous patch device, such as a glucose monitoring device. In some embodiments, an increased wearable life for the transdermal and/or subcutaneous device is provided, with greater protection, for example, up to 30 days of protection.

Some embodiments of an overlay securing patch may have a generally rectangular shape, including a major axis along a longer dimension of the rectangular shape. Such embodiments, if attached to a limb, may be oriented either with the major axis aligned parallel to the length of the limb, or may be oriented with the major axis aligned perpendicular to the length of the limb. Embodiments may be sized according to a wearable medical device being secured by the overlay securing patch. Some embodiments may include a transparent window, which may be useful, e.g., to observe the wearable medical device for any status indicators on it (e.g., LEDs, LCD message display, etc.). Embodiments of the overlay securing patch may be approximately 4 inches along the major axis, and have an exemplary surface area as shown in FIG. 1.

Embodiments in accordance with the present disclosure hold the wearable medical device securely. Leads (e.g., probes, sensors, etc.) from the wearable medical device typically will pierce epidermal layers of the patient's skin, therefore the wearable medical device must be held in place without motion relative to the patient's skin. The wearable medical device may continue to be held in place with an FDA-approved securing device, while embodiments supplement the FDA-approved securing device with an overlay device that provides additional adhesive surface areas.

In some embodiments, a message may be provided on the overlay securing patch, for example, an informative message to inform a caregiver or first responder as to the nature or purpose of the wearable medical device being secured by the overlay securing patch, or a message to notify a caregiver or first responder as to triage or other emergency instructions, or a message to notify caregiver or first responder emergency contact information, and so forth. Some embodiments may include a “peel away” type window, such that a surface underneath the peel away window includes text (e.g., instructions, legal notices, logo, etc.), and a top layer includes a relatively more fanciful and/or decorative display. The top layer may include a design selected by the patient or created by the patient. As discussed more fully below, informative content may also provide emergency instructions to a caregiver or first responder. Instructions may include, for example, steps for removal of the underlying device and/or remedial care steps. Remedial steps may include, for example, administer insulin.

Embodiments of an overlay securing patch may be partially transparent, partially opaque, or a combination of the two in different areas of the overlay securing patch.

Embodiments in accordance with the present disclosure may include custom shapes according to preferred design.

Embodiments in accordance with the present disclosure may include a written message intended for a target audience. For example, the written message may include an advertising message visible when the securing patch is worn by a patient, in exchange for providing the security patch to the patient for free, or a low cost, or a subsidized cost. The written message may promote giving away the securing patch to a target audience (e.g., patients, or medical service providers who may distribute the securing patch) in return for delivery of the written message contained on the securing patch.

In some embodiments, the written message may include one or more of: a public service message; an advertisement; an instruction about how to use a medical device being secured by the securing patch; a date of installation or first use of the medical device; a service date or expiration date of the medical device; an identification of the patient, a caregiver, an emergency point of contact, a next of kin; a listing of medical alert information about the patient (e.g., known allergies, whether the patient is epileptic, etc.), other textual message, and so forth.

In some embodiments, a printing on the securing patch may be decorative. For example, the printing may include a non-textual element such as an interesting graphic image (similar to a tattoo or henna tattoo), a comic character, a caricature, a sports team logo, a symbol (e.g., a yellow ribbon, an American flag, a cross, etc.), and so forth. In some embodiments, a printing on the securing patch may include an indication of a preferred orientation or direction of wearing either the securing patch or the underlying medical device. Still further aspects include symbols that have meaning, but are not limited to a particular language. Symbols may indicate danger or care instructions.

Embodiments in accordance with the present disclosure may be fabricated using a material such as a microporous rayon nonwoven fabric medical tape (e.g., 3M™ 1529 or equivalent). Such a material includes a white rayon nonwoven fabric backing with acrylic binder, coated with a pressure sensitive acrylate adhesive. The liner may be silicone treated, and polyethylene coated on one side, and including bleached Kraft paper. Such a material may be breathable and may provide a good surface to accept writing.

In some embodiments, a securing patch include or enclose additional sensors, e.g., a blood glucose sensor or other primary sensing device, piggybacked with additional secondary monitoring devices. The sensors may be situated within a single large chamber of the securing patch, or the securing patch may include more than one chamber such that at least one sensor is in a separate chamber from other sensors. The secondary monitoring devices may include sensors to measure states or parameters such as patient temperature as an indication of general health, insertion site temperature as a sign of infection, pulse rate, transdermal light transmission (e.g., to measure blood constituent such as iron), and so forth.

Embodiments may include a radio frequency identification (RFID) chip to store information such as patient identification, patient medication list and dosage, date of installation of the securing patch or wearable medical device, time of installation, geographic location of installation, ID of an installer, and so forth. The RFID chip information may be viewed, programmed, and/or updated from a smart device (e.g., iPhone). The RFID chip may be scanned, and information read from it used to access additional information on a protected website. In some embodiments, the RFID chip may store at least some measurements from the wearable medical device, and the stored measurements may be read from the RFID chip and uploaded to and aggregated with a remote data repository (e.g., a protected website). The secure website may be either a dedicated web site URL per patient, or a website portal to service multiple users together with usage of credentials and authentication to keep patient data private and accessible only to authorized persons, per applicable laws. A peel away portion of the label may be functional to reveal instructions for accessing a secure website in an emergency situation. The Instructions may include URL's and answers to logon and/or security queries.

Embodiments in accordance with the present disclosure may include an additional signal booster from primary sensor to receive signal and have a larger power source and antenna to transmit further or more clearly. This feature may facilitate connectivity of the securing patch with a base station located relatively farther away, e.g., one base station within a patient's house and accessible within either the entire house or within a room containing the base station.

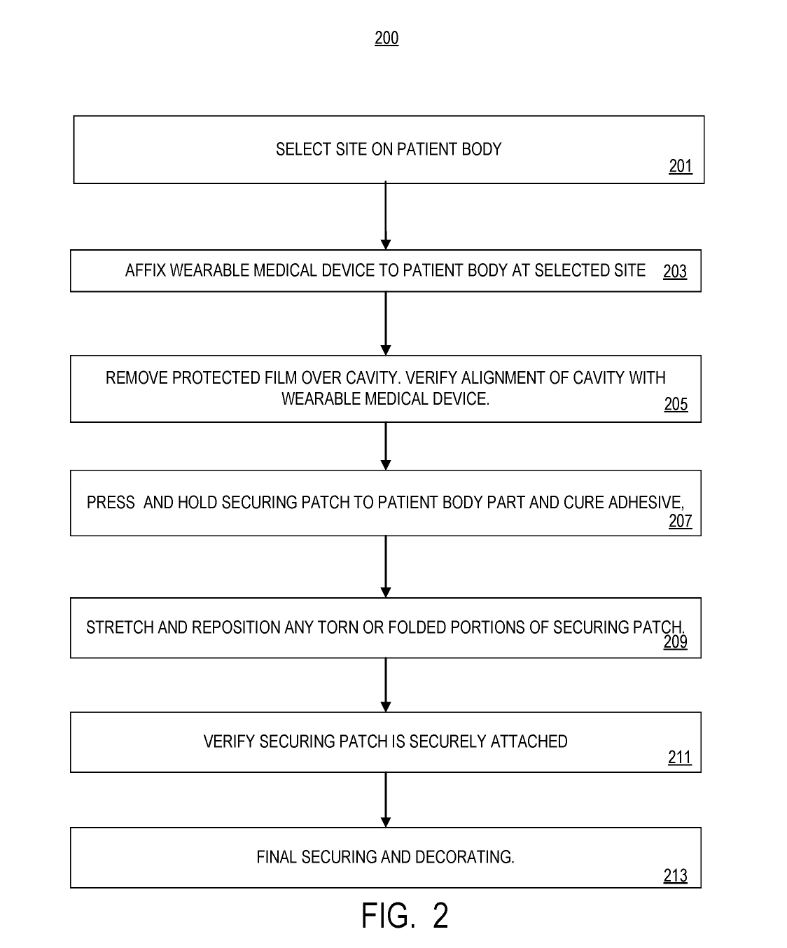

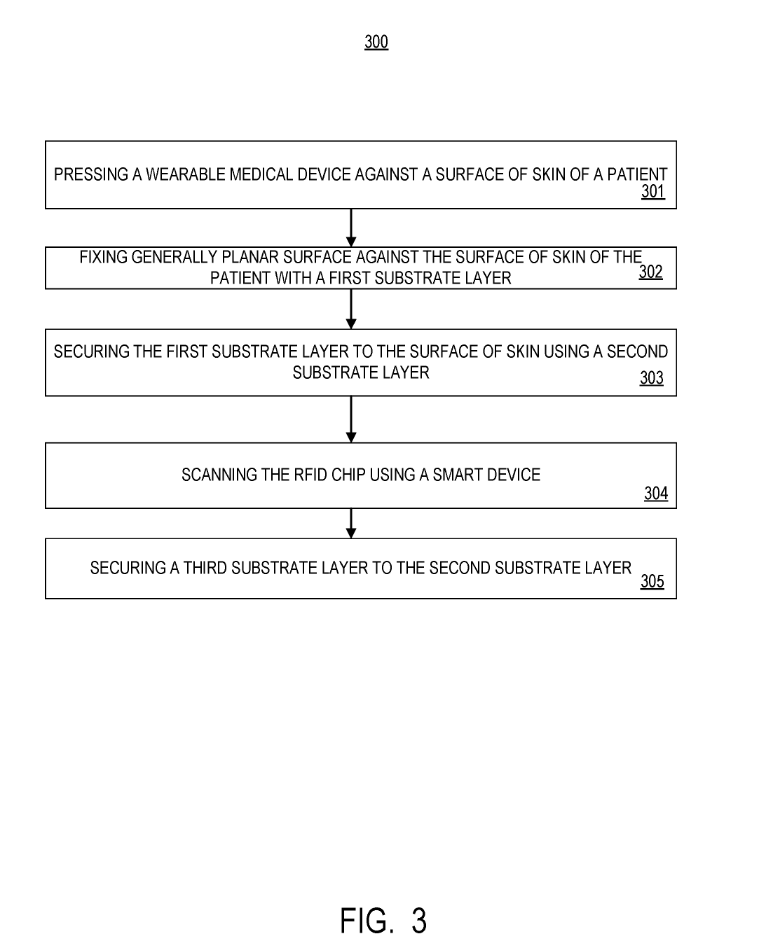

FIG. 2 illustrates a process 200 to install a securing patch for use with a wearable medical device such as a continuous glucose monitor (CGM) or insulin pump.

Process 200 begins with step 201, at which a site on the patient's body is selected for adhering the securing patch. A location should have good coverage both on the device and the surrounding skin areas. A location also should not be subject to excessive twisting or movement. This will allow the patch to be in a preferred position for securing the wearable medical device to the patient's body. For example, on a knee would not be a good location, but on a torso or abdomen might be a good location even though that location is subject to some movement as a patient stands up.

Next, control of process 200 moves to step 203, at which the wearable medical device should be affixed to the patient if it is not already affixed. Once the wearable medical device is affixed, the securing patch should be oriented such that the cavity in it is aligned with the wearable medical device, so the wearable medical device would fit in the cavity.

Next, control of process 200 moves to step 205, at which a protective film or layer covering an adhesive surface is removed at least enough to see the cavity in the securing device, and to verify that the cavity is being lined up with the wearable medical device as the securing device is brought near to the wearable medical device. If the protective film or layer is torn during this step, the torn portion should be folded back so it does not interfere with the rest of the adhesive surface.

Next, control of process 200 moves to step 207, at which the securing patch is pressed to the skin and held while the remainder of the protective film or layer is removed. The securing patch should be aligned with the wearable medical device as the protective film or layer is removed. Pressure should be applied to the entire securing patch, without excessive stretching of either the wearable medical device or the securing patch before the securing patch fully adheres. In some embodiments, a heat-activated adhesive may be used, which uses body heat to activate and cure the adhesive. For such embodiments, the adhesive layer of the securing patch should be pressed against the patient's body and held still for at least about 30 seconds. Curing of the adhesive may be facilitated by application of a moderate amount of heat, such as from a hair dryer, both before and during the pressing of the securing patch against the patient's skin.

Next, control of process 200 moves to optional step 209, at which if a portion of the patch had torn or had folded upon itself, the securing patch may be stretched slightly to free and reposition the folded or torn portion, and then reposition the stretched securing patch over the wearable medical device.

Next, control of process 200 moves to step 211, at which all edges and corners are double-checked for a secure adhesion, and additional pressure and/or heat applied if and where necessary.

Next, control of process 200 moves to step 213, at which final securing and decoration can be made.

Embodiments should be cared for on a daily basis in order to maintain an attractive appearance. After activities that might cause the securing patch to become wet (e.g., showering, watersport activities, or gentle cleaning), edges and corners of the securing patch should be checked that they still have good adhesion, and push down or re-adhere where necessary. The embodiments should be patted dry rather than be rubbed dry.

A securing patch typically can be used for at least a week before it should be removed and replaced. If wishing to remove a securing patch, the center can be grasped and pulled up at an angle and torn off while trying not to pull up the sensor tape adhesion. Embodiments are water-resistant and stretchable, so there may be some adhesive residue on the patient's skin after removal. Lotions, oils and hand sanitizer with some rubbing action may be used to remove any adhesive residue. However, in exemplary embodiments, the securing patch may be waterproof (i.e., the securing patch should comprise a water-impermeable barrier).

Referring now to FIG. 3, an exemplary method for securing a wearable medical device against or into a patient's skin 300 is shown. The patient may be human 101. At step 301, a wearable medical device comprising a generally planar surface is pressed against a surface of skin of a patient. The patient comprises a skin and a body. The wearable medical device may be an insulin pump, a CGM, or other device to monitor the amount of a chemical in the patient's body, or to deliver drug dosages into the patient's body. The wearable medical device comprises a generally planar surface.

At step 302, the generally planar surface of the wearable medical device is fixed against the surface of skin of the patient using a first substrate layer. The first substrate layer comprises a flexible material and has a surface area sufficiently large to encompass the wearable medical device. In some embodiments, the wearable medical device is specifically an insulin pump. In such embodiments, the insulin pump comprises a subcutaneous probe, and thus the probe must be inserted into the patient's skin at step 302.

At step 303, the first substrate layer is secured to the patient's skin using a second substrate layer. The second substrate layer comprises an adhesive coating and at least one medical information quantum. The second substrate layer has a surface area between approximately 3.0 square inches and 4.0 square inches. By way of non-limiting example, the medical information quantum may comprise: patient identification, wherein the patient identification comprises one or more of: a name of the patient, an address of the patient, a medication list of the patient, or an RFID chip. In some embodiments, the second substrate layer comprises a decorative printing on a face of the second substrate layer.

At step 304, if the medical information quantum comprises an RFID chip, then the RFID chip may be read by any device capable of doing so, such as a smart device, which comprises a processor, software executable on command, and a display in logical connection with the processor. When so read, the RFID chip may cause to appear on the display of the smart device RFID chip information. RFID chip information may comprise the aforementioned patient identification. In some embodiments, the RFID chip information may comprise device identification, wherein the device identification comprises one or more of: a serial number of the wearable medical device, a manufacturer of the wearable medical device, a time of installation of the wearable medical device, a geographic location of installation of the wearable medical device, identification information related to an installer of the wearable medical device, an expiration date of the wearable medical device, or a recommended time of removal of the wearable medical device. In other embodiments, the RFID chip information comprises measurements from the wearable medical device, wherein the measurements comprise one or more of: an amount of a chemical in the patient's body, an amount of medicine administered to the patient over a defined interval of time through the wearable medical device, or a pulse. In other embodiments, the RFID chip contains a uniform resource locator, which points to a website having patient or device information.

At step 305, a third substrate layer is secured to the second substrate layer. The third substrate layer comprises an adhesive and has a surface area of between approximately 4.0 square inches and 11.0 square inches. In some embodiments, the third substrate layer comprises a water-impermeable barrier. In additional embodiments, the third substrate layer is generally transparent, to allow easy viewing of the second substrate layer. In some embodiments, the second substrate layer comprises a written message on a surface of the second substrate layer, wherein the written message comprises one or more of: a public service message, an advertisement, an instruction about usage of the wearable medical device, a date of installation or first use of the wearable medical device, a service date or expiration date of the wearable medical device, an identification of the patient, a caregiver, an emergency point of contact, a next of kin, or a listing of medical alert information about the patient. In some embodiments, the second substrate layer comprises a decorative printing on a face of the second substrate layer.

A number of embodiments of the present disclosure have been described. While this specification contains many specific implementation details, there should not be construed as limitations on the scope of any disclosures or of what may be claimed, but rather as descriptions of features specific to particular embodiments of the present disclosure. While embodiments of the present disclosure are described herein by way of example using several illustrative drawings, those skilled in the art will recognize the present disclosure is not limited to the embodiments or drawings described. It should be understood the drawings and the detailed description thereto are not intended to limit the present disclosure to the form disclosed, but to the contrary, the present disclosure is to cover all modification, equivalents and alternatives falling within the spirit and scope of embodiments of the present disclosure as defined by the appended claims.

The headings used herein are for organizational purposes only and are not meant to be used to limit the scope of the description or the claims. As used throughout this application, the word “may” is used in a permissive sense (i.e., meaning having the potential to), rather than the mandatory sense (i.e., meaning must). Similarly, the words “include”, “including”, and “includes” mean including but not limited to. To facilitate understanding, like reference numerals have been used, where possible, to designate like elements common to the figures.

The phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The term “a” or “an” entity refers to one or more of that entity. As such, the terms “a” (or “an”), “one or more” and “at least one” can be used interchangeably herein. It is also to be noted the terms “comprising”, “including”, and “having” can be used interchangeably.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while method steps may be depicted in the drawings in a particular order, this should not be understood as requiring that such operations be performed in the particular order shown or in a sequential order, or that all illustrated operations be performed, to achieve desirable results.

Certain features that are described in this specification in the context of separate embodiments can also be implemented in combination in a single embodiment. Conversely, various features that are described in the context of a single embodiment can also be implemented in combination in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the subject matter have been described. Other embodiments are within the scope of the following claims. In some cases, the actions recited in the claims can be performed in a different order and still achieve desirable results. In addition, the processes depicted in the accompanying figures do not necessarily require the particular order show, or sequential order, to achieve desirable results. In certain implementations, multitasking and parallel processing may be advantageous. Nevertheless, it will be understood that various modifications may be made without departing from the spirit and scope of the claimed disclosure.

In certain implementations, multitasking and parallel processing may be advantageous. Nevertheless, it will be understood that various modifications may be made without departing from the spirit and scope of the claimed disclosure.

What is claimed is:

1. A method for securing a wearable medical device against or into a patient's skin, the method comprising the steps of:
a) pressing a wearable medical device comprising a generally planar surface against a surface of skin of a patient, wherein the patient comprises a skin and a body;
b) fixing the generally planar surface against the surface of skin of the patient with a first substrate layer, wherein the first substrate layer comprises a flexible material and has a surface area sufficiently large to encompass the wearable medical device; and
c) securing the first substrate layer to the surface of skin of the patient using a second substrate layer, wherein the second substrate layer comprises an adhesive coating and at least one medical information quantum and has a surface area between approximately 3.0 square inches and 4.0 square inches.

2. The method of claim 1, wherein the medical information quantum comprises an RFID chip.

3. The method of claim 2, further comprising the steps of: scanning the RFID chip using a smart device, wherein the smart device comprises a processor, software executable on command, and a display in logical connection with the processor, to receive RFID chip information into the smart device; and displaying the RFID chip information on the display.

4. The method of claim 3, wherein the RFID chip information comprises patient identification, wherein the patient identification comprises one or more of: a name of the patient, an address of the patient, or a medication list of the patient.

5. The method of claim 3, wherein the RFID chip information comprises device identification, wherein the device identification comprises one or more of: a serial number of the wearable medical device, a manufacturer of the wearable medical device, a time of installation of the wearable medical device, a geographic location of installation of the wearable medical device, identification information related to an installer of the wearable medical device, an expiration date of the wearable medical device, or a recommended time of removal of the wearable medical device.

6. The method of claim 3, wherein the RFID chip information comprises measurements from the wearable medical device, wherein the measurements comprise one or more of: an amount of a chemical in the patient's body, an amount of medicine administered to the patient over a defined interval of time through the wearable medical device, or a pulse.

7. The method of claim 6, wherein the RFID chip information comprises a uniform resource locator pointing to a website.

8. The method of claim 1, wherein the second substrate layer comprises a decorative printing on a face of the second substrate layer.

9. The method of claim 1, further comprising the step of: securing a third substrate layer to the second substrate layer, wherein the third substrate layer comprises an adhesive and has a surface area of between approximately 4.0 square inches and 11.0 square inches.

10. The method of claim 9, wherein the third substrate layer comprises a water-impermeable barrier.

11. The method of claim 9, wherein the third substrate layer is generally transparent.

12. The method of claim 9, wherein the second substrate layer comprises a written message on a surface of the second substrate layer, wherein the written message comprises one or more of: a public service message, an advertisement, an instruction about usage of the wearable medical device, a date of installation or first use of the wearable medical device, a service date or expiration date of the wearable medical device, an identification of the patient, a caregiver, an emergency point of contact, a next of kin, or a listing of medical alert information about the patient.

13. The method of claim 9, wherein the second substrate layer comprises a decorative printing on a face of the second substrate layer.

14. The method of claim 9, wherein the wearable medical device comprises an insulin pump.

15. The method of claim 14, wherein the wearable medical device further comprises a subcutaneous probe, and wherein the method further comprises inserting the subcutaneous probe into the patient's skin prior to the step of securing the first substrate layer.

16. A method for installing a securing patch for use with a wearable medical device, the method comprising the steps of:
a) selecting a site on a patient's body, wherein the site comprises a surface area of at least 4.0 square inches;
b) affixing the wearable medical device to the site on the patient's body;
c) placing a securing patch on the wearable medical device, wherein the securing patch comprises a protective film covering an adhesive, a writable portion, and a hollow chamber of sufficient depth to enclose any supracutaneous portion of the wearable medical device;
d) pressing the securing patch to the site;
e) holding the securing patch on the site while removing the protective film;
f) curing the adhesive;
g) applying heat and/or pressure to any uncured portion of the adhesive; and
h) placing one or more medical information quanta on the writable portion.

17. The method of claim 16, wherein the adhesive comprises a heat-activated adhesive with a curing temperature between 90.0° Fahrenheit and 101.0° Fahrenheit.

18. The method of claim 16, wherein the securing patch further comprises a design portion, wherein the design portion comprises one or more of: a graphic image, a comic character, a caricature, a sports team logo, or a symbol.

19. The method of claim 16, wherein the one or more medical information quanta comprises one of: a textual message, an instruction, an advertisement, an identification of the patient, a caregiver, an emergency point of contact, a next of kin, a listing of medical alert information about the patient, or a legal notice.

20. An adhesive patch to secure an insulin pump to a patient's body, the adhesive patch comprising:
a) a flexible first substrate layer, wherein the first substrate layer is molded to include a chamber sufficiently sized to enclose any supracutaneous portion of the insulin pump;
b) an adhesive second substrate layer to secure the first substrate layer to the patient's skin, wherein the second substrate layer has a surface area between approximately 3.0 square inches and 4.0 square inches, and comprises a first face comprising an adhesive, and a second face comprising a visual display, wherein the visual display comprises one or more of: a graphic image, a comic character, a caricature, a sports team logo, a symbol, a textual message, an instruction, an advertisement, an identification of the patient, a caregiver, an emergency point of contact, a next of kin, a listing of medical alert information about the patient, legal notice, a uniform resource locator, or an RFID chip; and
c) a transparent third substrate layer having a surface area of between approximately 4.0 square inches and 11.0 square inches, and comprising a water-impermeable barrier.
